(19) United States
(12) Patent Application Publication
(10) Pub. No.: US 2010/0168793 A1
(43) Pub. Date: Jul. 1, 2010

(54) SYSTEM COMPRISING A PLATE AND PEDICLE SCREWS, AND ITS APPLICATIONS

(75) Inventor: Jérôme Levieux, Bellevue (CH)

Correspondence Address: YOUNG & THOMPSON, 209 Madison Street, Suite 500, Alexandria, VA, 22314, US

(73) Assignee: SPINEART SA, Meyrin (CH)

(21) Appl. No.: 12/160,790

(22) PCT Filed: Jan. 10, 2007
(86) PCT No.: PCT/FR2007/000040

§ 371 Date: Jul. 14, 2008

(30) Foreign Application Priority Data
Jan. 13, 2006 (FR) 0600332

(51) Int. Cl. A61B 17/70

(52) U.S. Cl. 606/246

(57) ABSTRACT

A lumbar plate, which can be implanted by a posterior approach in order to partially immobilize adjacent vertebrae, includes two holes (13) which are spaced apart and through which screws (2) can be introduced into adjacent lumbar vertebrae, the lumbar plate (12) including, between the two holes (13), an extensible and compressible zone made of elastic material.

The present invention relates to a plate and pedicle screws system and applications thereof.

Conditions of the lumbar spine are treated in different ways. One of these is to immobilize several vertebrae in order to effect an arthrodesis of the joints concerned, generally by fusing them using bone grafting.

One technique is to fit pedicle screws joined together by a titanium or stainless steel plate.

Immobilizing vertebrae has several disadvantages, two of which are as follows: the joints adjacent to the sites of immobilization are far more highly stressed, and the patient's ability to move is diminished.

That is why so-called “non-fusion” techniques are emerging. These involve reestablishing the anatomical distances and movements in order to make the joints operate as normally as possible again.

One technique is to use an anterior route to fit a prosthesis in place of the afflicted disk, as described, for example in US-A-6 368 350. This recent technique of fitting via an anterior route has the disadvantage of being the preserve of a restricted number of surgeons trained in and accustomed to managing the associated risks.

Another technique is to use a posterior route to fit pedicle screws connected by a ligament and a silicone tube as described for example in FR-A-2 777 449. This technique has the advantages of the posterior route, but, on the one hand, the fitting technique is complicated because the surgeon has to use a great many instruments in order to tension the elements and, on the other hand, if the surgeon wishes to immobilize one joint and perform a “non-fusion” technique on the adjacent joint, this type of system will not allow him to do so.

It would therefore be desirable to have a system that overcame these disadvantages.

Now, after extensive research, the applicant company has developed a system of pedicle screws, which are therefore fitted to the vertebra via the posterior route, that has proven to be satisfactory.

The purpose of the subject matter of the invention is to overcome these disadvantages by proposing a system of pedicle screws joined together by a lumbar plate part of which is made of an elastic material so as to allow the vertebrae some movement while at the same time maintaining the same operating procedure as used for immobilizing vertebrae as this procedure is the one at which all surgeons specializing in spinal conditions are adept.

Let us remember here that the devices of the invention are aimed at the field of posterior lumbar plates and allow two lumbar vertebrae to be joined together via a posterior route, and not, for example, at the field of anterior, for example cervical, plates. Further, the objective of the invention is to avoid fusing two lumbar vertebrae together, thereby allowing natural anatomical movements, whereas the objective of anterior plates is to fuse two cervical vertebrae together.

In consequence, anterior plates are generally 14 to 30 mm wide, 1 to 4 mm thick and 20 to 80 mm long, whereas the plates such as those of the present invention are generally 8 to 14 mm wide, 6 to 14 mm thick and 30 to 200 mm long. In addition, since anterior cervical plates are designed for fusion, the vertebrae have not to move and any elastic region provided is there solely to maintain tension on the graft so that it takes better. The dimensions and the mechanical properties of the elastic part will therefore be completely different from those of the devices of the invention. For anterior cervical plates, the material selected will be stiff and small in size, allowing only very small movements of the order of a few tenths of an mm.

The devices of the invention, which are used to join two lumbar vertebrae together via a posterior route are also fundamentally different from, for example, a vertebral ligament prosthesis. They perform a different function because a ligament prosthesis works only in extension and in rotation whereas a posterior lumbar plate works in bending, extension, compression and rotation. Numerous structural differences will be evident to those skilled in the art. In particular, the thickness of a ligament prosthesis will of course be fairly small because it is not designed to work in bending and in extension like posterior lumbar plates. In addition, fitting a ligament prosthesis in the anterior part does not allow the use of thick plates. The width of the two devices is also very different. Using the anterior route, just one “ligament” is fitted to the anterior part of the body, whereas two posterior lumbar plates have to be fitted, and these need to be installed via a posterior route and will therefore need to be narrower.

The degrees of deformation of the plate will also differ widely because an anterior ligament deforms very little (and then only in tension and in torsion) whereas a posterior lumbar plate flexes to a very great extent during natural movements. It therefore follows that the volume and composition of the flexible materials need to be very different.

This is why the subject of the present application is a lumbar plate that can be implanted for partially immobilizing adjacent lumbar vertebrae via a posterior route comprising two spaced-apart holes to allow screws to be inserted into adjacent vertebrae, said lumbar plate comprising, between the two holes an extensile and compressible region made of an elastic material.

The implantable plate is preferably of elongate shape, for example of oval and in particular oblong, shape.

The holes are preferably made in metal inserts provided in the plate. These inserts may conventionally be secured to the plate through the use of shaped edges. Inserts with shaped edges are illustrated hereinafter in the figures.

In preferred embodiments of the invention the region made of an elastic material represents most of the plate and the holes are made in metal inserts provided in a plate made of an elastic material.

In other preferred embodiments of the invention, the holes are made in metal strips joined together by a band made of an elastic material.

In still further preferred embodiments of the invention, the implantable plate is made mainly of a metallic material, and one of the holes is made in a metal insert fixed to the implantable plate using a ring made of an elastic material. In this embodiment, a metallic material represents most of the component.

In this application and in that which follows, the term “hole” denotes, in particular, a simple hole through which a screw can pass, but advantageously denotes a countersunk hole that hugs the shape of the head of the screw and allows the screw to pass.

While the holes may be circular, they are preferably oblong in order to suit different spacings.

In an alternative form of embodiment of the invention, the implantable plates according to the invention comprise three substantially aligned holes for fixing to three adjacent vertebrae.

In an alternative form of embodiment of the invention, the above plates comprise a rigid plate installed between two adjacent holes. Thus, the immobilizing of one region and the stabilizing of an adjacent region can be combined.

The metal inserts, plates or strips are made of implantable materials, preferably titanium or stainless steel or alloys thereof.

The extensile and compressible region made of an elastic material is preferably made of a material of the polyurethane type, particularly of a polyurethane like the one marketed by POLY MEDICA INDUSTRIES Inc (Massachusetts) under the trade name CHRONOFLEX®.

When the region made of an elastic material represents most of the implantable plate, and the holes are made in metal inserts provided in a plate made of an elastic polyurethane material, and the plate is oblong in shape,
the plate may be 3 to 14 mm, preferably 4 to 13 mm, particularly 5 to 12 mm, and most especially 6 to 11 mm, thick.
the plate may be 5 to 18 mm, preferably 6 to 16 mm, particularly 7 to 15 mm, and quite especially 8 to 14 mm, wide.
the plate may be 20 to 70 mm, preferably 30 to 60 mm, particularly 35 to 55 mm, and quite especially 40 to 50 mm, long, in the case of a plate implanted across two adjacent vertebrae.
the plate may be 50 to 100 mm, preferably 60 to 90 mm, particularly 65 to 85 mm, and most especially 70 to 80 mm long, in the case of a plate implanted across three adjacent vertebrae.

The length of the plate depends on the number of vertebrae affected and may vary from 30 to 200 mm.

Of appropriate dimensions, the elastic material is capable of allowing the plate to mimic the movements of the joint. For example, if a person bends forward, the disk located between L4 and L5 will need to squash by about 2 mm at the front; if the individual bends to his or her right, this disk will need to squash by about 1.5 mm on its right-hand side. The elastic component will therefore have the thickness and width needed to allow preferably the maximum amplitudes required for anatomical movements. The elastic component may also have the thickness and width needed to allow at least 50%, advantageously at least 70%, particularly at least 80%, and most especially at least 90% of the maximum amplitudes needed for an anatomical movement.

An implantable plate of the invention is produced by using conventional methods and, in particular, by overmolding elastic material over the metallic base or bases (inserts, plates or strips).

When a less flexible implantable plate is to be produced, a plate is advantageously made that is greater in width and thickness. If, on the other hand, a more flexible implantable plate is to be produced, then a plate of lesser width and/or thickness, at least in the part between the two holes, is made.

An implantable plate of the invention can be sterilized just before it is fitted to vertebrae or may preferably come wrapped in sterile form. Screws are the preferred method of fitment.

This is why a further subject of the present application is an assembly or kit comprising an implantable plate as hereinabove together with the appropriate pedicle screws. Let us remember here that pedicle screws are usually 3.5 to 5 cm long with a diameter ranging from 0.5 to 0.7 cm and are screws designed for fastening into the pedicles located in the posterior part of the vertebrae.

The implantable plates that are the subject of the present invention have highly beneficial properties and qualities. They are able in particular to stabilize the lumbar spine while at the same time permitting anatomical movement.

In addition, when treating several sites, the plate can have a rigid part intended to immobilize one joint and a flexible part intended for a “non-fusion” technique on the adjacent joint.

These properties are illustrated hereinafter in the experimental part. They justify the use of the implantable plates according to the above-described invention particularly the surgical treatment of degenerative disk disease.

This is why a further subject of the present application is a method for the surgical treatment of degenerative disk disease in which method at least one abovementioned implantable plate is fixed via a posterior route to at least two adjacent vertebrae using screws.

The preferred embodiments of the implantable plates described hereinabove also apply to the other subjects of the invention mentioned hereinabove, particularly to the assemblies or kits and methods described hereinabove.

The invention will be better understood with reference to the attached drawings in which:

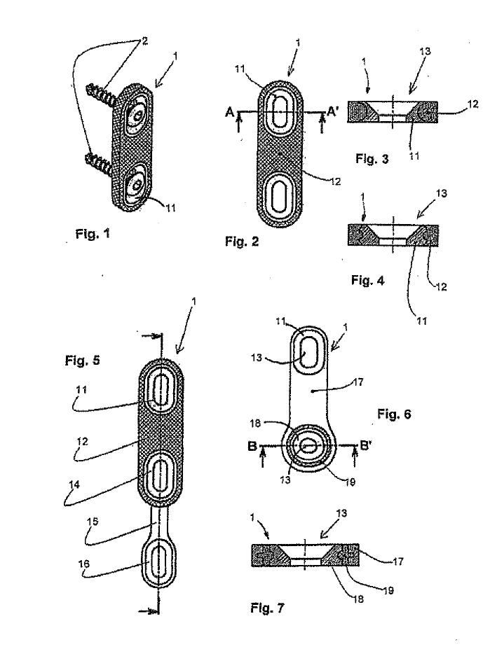

FIG. 1 depicts a perspective view of an implantable plate and the screws therefor;

FIG. 2 depicts another view of an implantable plate, from above;

FIG. 3 depicts a section on AA′ of FIG. 2 (on a larger scale);

FIG. 4 depicts a section on AA′ of FIG. 2 (on a larger scale) (alternative form);

FIG. 5 depicts a view of an implantable plate from above;

FIG. 6 depicts a view of an implantable plate from above;

FIG. 7 depicts a section on BB′ of FIG. 6 (on a larger scale).

FIG. 1 shows a plate according to the invention denoted overall by 1, of oblong shape, essentially made of an elastic polyurethane plastic material because only two inserts 11 are made of metal. The dimensions of this plate are as follows: thickness 8 mm, width 11 mm, length 50 mm. Two screws 2 can be used to fix this plate 1 to two adjacent vertebrae. The heads of the screws 2 are inserted in two holes made in the inserts 11.

FIG. 2 shows that two identical inserts 11 are provided in the plate 12. These inserts 11 each have at their center an oblong countersink 13 that can be seen better later in FIG. 3.

FIG. 3 shows the concave shape of the edges of the insert 11 that anchor it within the plate 12 with convex bulges. The hole 13 made through the insert 11 comprises a countersunk part capable of collaborating with the head of a screw 2 to prevent this screw head from protruding or protruding excessively above the plate 1. The holes 13 are elongate to suit different vertebra spacings.

FIG. 4 shows an alternative form of embodiment of the edges of the insert, which in cross section have a wavy overall shape.

FIG. 5 depicts an insert capable both of immobilizing a joint between two vertebrae and of effecting the “non-fusion” technique on the joint between two adjacent vertebrae. Thus, one of the inserts 11 is an insert similar to those described hereinabove and the second insert 14 is extended by a metal tab 15 equipped with a hole 16. The spacing between the inserts 11 and 14 is identical to the spacing between the inserts 14 and 16. The assembly formed of the inserts 14, 16 and of the tab 15 is produced as a single piece.

In FIG. 6, the plate 17 is essentially made of metal. One of the inserts 18 is separated from the plate 17 by an annular sleeve 19 made of polyurethane. The hole 13 through this insert 18 is circular. Just one elongate hole is enough to allow a wide range of adjustment.

FIG. 7 shows the insert 18 sheathed with the polyurethane ring 19 driven into the plate 17. It is also possible to see an alternative form of embodiment of the edges of the insert 18 which, in cross section, have a cupped overall shape and the tab-slot collaboration of the external edges of the polyurethane ring 19.

1. A lumbar plate that can be implanted via a posterior route for partially immobilizing adjacent vertebrae comprising two spaced-apart holes (13) to allow screws (2) to be inserted into adjacent lumbar vertebrae, said lumbar plate (12) comprising, between the two holes (13) an extensile and compressible region made of an elastic material.

2. An implantable plate as claimed in claim 1, characterized in that the holes (13) are made in metal inserts (11) provided in the plate (12).

3. An implantable plate as claimed in claim 1, characterized in that the region made of an elastic material represents most of the plate (12) and the holes (13) are made in metal inserts (11) provided in a plate (12) made of an elastic material.

4. An implantable plate as claimed in claim 1, characterized in that the holes (13) are countersunk.

5. An implantable plate as claimed in claim 1, characterized in that the holes (13) are oblong.

6. An implantable plate as claimed in claim 1, characterized in that it comprises three substantially aligned holes (13) that can be fixed to three adjacent vertebrae.

7. An implantable plate as claimed in claim 6, characterized in that it comprises a rigid plate (15) installed between two adjacent holes (14, 16).

8. An implantable plate as claimed in claim 1, characterized in that the extensile and compressible region made of an elastic material is made of a material of the polyurethane type.

9. An implantable plate as claimed in claim 1, characterized in that the region made of an elastic material is of appropriate dimensions and is selected to allow the plate to mimic the movements of the joint.

10. An implantable plate as claimed in claim 1, characterized in that the region made of an elastic material represents most of the implantable plate, in that the holes (13) are made in metal inserts (11) provided in a plate (12) made of an elastic polyurethane material, and in that the plate (12) is of oblong shape, and
either the plate (12) is 4 to 13 mm thick,
or the plate (12) is 6 to 16 mm wide,
or the plate (12) is 35 to 55 mm long in the case of plate implanted across two adjacent vertebrae,
or the plate (12) is 65 to 85 mm long in the case of a plate implanted across three adjacent vertebrae.

11. An assembly comprising an implantable plate as defined in claim 1 and suitable pedicle screws (2).

12. An implantable plate as claimed in claim 2, characterized in that the region made of an elastic material represents most of the plate (12) and the holes (13) are made in metal inserts (11) provided in a plate (12) made of an elastic material.

13. An implantable plate as claimed in claim 2, characterized in that the holes (13) are countersunk.

14. An implantable plate as claimed in claim 2, characterized in that the holes (13) are oblong.

15. An implantable plate as claimed in claim 2, characterized in that it comprises three substantially aligned holes (13) that can be fixed to three adjacent vertebrae.

16. An implantable plate as claimed in claim 2, characterized in that the extensile and compressible region made of an elastic material is made of a material of the polyurethane type.

17. An implantable plate as claimed in claim 2, characterized in that the region made of an elastic material is of appropriate dimensions and is selected to allow the plate to mimic the movements of the joint.

18. An implantable plate as claimed in claim 2, characterized in that the region made of an elastic material represents most of the implantable plate, in that the holes (13) are made in metal inserts (11) provided in a plate (12) made of an elastic polyurethane material, and in that the plate (12) is of oblong shape, and
either the plate (12) is 4 to 13 mm thick,
or the plate (12) is 6 to 16 mm wide,
or the plate (12) is 35 to 55 mm long in the case of plate implanted across two adjacent vertebrae,
or the plate (12) is 65 to 85 mm long in the case of a plate implanted across three adjacent vertebrae.

19. An assembly comprising an implantable plate as defined in claim 2 and suitable pedicle screws (2).

20. An implantable plate as claimed in claim 3, characterized in that the holes (13) are countersunk.
